SEQUENCE LISTING (1 sequence)

SEQ ID NO: 1 - protein, 214 aa, unidentified
RVQPTESIVRFPNITNLCPFGEVFNATRFASVYAWNRKRISNCVADYSVLYNSASFSTFKCYTKLNDLCFTNVYADSFVIRGDEVRQIAPGQTGKIADYNYKLPDDFTGCVIAWNSNNLDSKVGGNYNYLYRLFRKSNLKPFERDISTAGSTPCNGVEGFNCYFPLQSYGFQPTNGVGYQPYRVVVLSFELLHAPATVCGPKKSTNVKNKCVNF

(19) United States
(12) Patent Application Publication
(10) Pub. No.: US 2024/0418720 A1
(43) Pub. Date: Dec. 19, 2024

(54) ELECTRON-TUNNELING OR HOPPING BASED AERIAL VIRAL DETECTOR

(71) Applicant: The Board of Trustees of the University of Illinois, Urbana, IL (US)

(72) Inventors: Vikas Berry, Chicago, IL (US); Sungjoon Kim, Chicago, IL (US); Roshan Nemade, Chicago, IL (US); Sheldon Cotts, Chicago, IL (US)

(21) Appl. No.: 18/743,238

(22) Filed: Jun. 14, 2024

Related U.S. Application Data
(60) Provisional application No. 63/521,465, filed on Jun. 16, 2023.

(51) Int. Cl. G01N 33/569; G01N 27/04; G01N 27/327; G01N 33/00

(52) U.S. Cl. CPC G01N 33/56983; G01N 27/04; G01N 27/3278; G01N 33/0036; G01N 2333/11; G01N 2333/135; G01N 2333/165

(57) ABSTRACT

In one aspect, the disclosure relates to an aerial viral detection device comprising a sensor cartridge comprising at least one sensor, the at least one sensor comprising a self-hydrating hygroscopic surface and an integrated conductive polymer or nanoparticle network. In one aspect, the device further comprises a conductive component and a binding agent, the binding agent binding the conductive component to the hygroscopic surface. The aerial viral detection device further comprises an active element and a control element disposed in the sensor cartridge, wherein the control element includes all features of the active element with the exception of a molecular receptor such as an antibody. The disclosed devices, systems, and methods can rapidly detect COVID-19, influenza, RSV, and/or other infectious agents from air samples within a room in real time. The system disclosed herein can operate on the principle of electron hopping to detect viral particles with high sensitivity.

### CROSS REFERENCE TO RELATED APPLICATIONS

This application claims the benefit of U.S. Provisional Application No. 63/521,465 filed on Jun. 16, 2023, which is incorporated herein by reference in its entirety.

### CROSS-REFERENCE TO SEQUENCE LISTING

This application contains a sequence listing filed in ST.26 format entitled “320903-1390_Sequence_Listing.xml” created on Apr. 29, 2024, and having a file size of 2, 198 bytes. The content of the sequence listing is incorporated herein in its entirety.

### BACKGROUND

The COVID-19 pandemic has led to over 700 million infections and over 7 million lives lost through a high rate of airborne infections. Current aerial virus detection techniques collect samples from air intermittently and either send the sample for processing at a testing facility or process the samples automatically. A typical system is very costly, at over $5000.

The COVID-19 pandemic led to a sizeable global GDP contraction of 3.9% from 2019 to 2020, or about 3 trillion dollars. This is attributed to the cessation of business operations, employee productivity loss, and the closure of global commerce. There was widespread fear and panic over mortality rates and the unknown method of transmission during the early stages of the pandemic. These unknowns led to the enforcement of mask mandates, social distancing and occupancy reduction protocols, sanitization requirements, and vaccine development to curb the spread of the disease. Although these implementations were critical in fighting the pandemic and saving lives, they were all reactionary strategies that resulted in economic damage and loss of human lives. Crucial systems, devices, and information related to pathogen tracking are currently lacking. The COVID-19 pandemic led to significant disruptions in all economic sectors, including manufacturing (with a 20% decrease in 2020), energy (with a 10% decrease in 2020), mining, retail, finance, healthcare, and construction (with 25% of projects put on hold in 2020). Further, 10% people are reporting long COVID showing prolonged symptoms, which will increase the burden even more.

Influenza is responsible for about 5 million (41 million in US) severe cases each year, resulting in 650,000 deaths globally (52,000 in US). 70-80% of deaths occurring in the United States is of adults over the age of 65. Pregnant women, young children, individuals with underlying conditions, and healthcare workers are also at higher risk. Despite widespread vaccination for 65 years, in 2017, influenza caused an economic burden of $11.2 billion (before the COVID pandemic). This burden included loss in productivity and medical costs. Respiratory syncytial virus (RSV) infects 64 million individuals annually, resulting in 160,000 deaths worldwide.

Respiratory-tract viruses are predominantly spread through the release of droplets and aerosols from coughing, sneezing, talking, and breathing. The flow dynamics of these particles are influenced by various forces, such as inertia, gravity, evaporation, and convection, while following the laws of mass, angular momentum, and energy conservation. The density of particles, size distribution, and viral load of droplets are important parameters in their movement through the air. COVID patients shed between 3,900 and 5×10⁴ virus particles per hour while breathing and talking, while more severe patients can shed up to 3.6×10⁵ particles per hour. Influenza patients, on the other hand, shed approximately 7.5×10⁴ influenza virus particles per hour, while breathing. The presence of infected individuals in an enclosed room can easily infect others in the same space.

Every day, millions of individuals are exposed to aerial viruses without any prior knowledge or warning. Early detection of viral infections is critical to reducing mortality rates. Aerial detection of viruses provides the best opportunity for early detection thereby impacting the basic reproduction number (R0). However, the currently available methods are based on polymerase chain reaction (PCR) or quantitative PCR (qPCR) techniques, which require constant use of costly reagents (irrespective of result) and maintenance of expensive, bulky equipment, making them impractical for routine use by general population. While there are non-aerial virus-antigen-detectors (lateral-flow assays (LFA)), there is no antigen-based aerial virus detector. Current solutions for determining if a pathogen is present require travel and/or logistics costs regardless of whether test results are ultimately positive or negative. Such solutions produce excessive amounts of chemical and/or biological waste. Furthermore, detection and response are delayed, which can result in a critical time loss in alerting any occupants of a building of a hazardous and/or contagious environment.

Despite advances in pathogen detection research, there is still a scarcity of methods for instantaneous detection of viruses that are inexpensive, fast, accurate, and do not result in the production of large amounts of waste. These needs and other needs are satisfied by the present disclosure.

### SUMMARY

In accordance with the purpose(s) of the present disclosure, as embodied and broadly described herein, the disclosure, in one aspect, relates to an aerial viral detection device comprising a sensor cartridge comprising at least one sensor, the at least one sensor comprising a self-hydrating hygroscopic surface and an integrated conductive polymer or nanoparticle network. In one aspect, the aerial viral detection device further comprises a conductive component and a binding agent, the binding agent binding the conductive component to the hygroscopic surface. In another embodiment, the aerial viral detection device further comprises an active element and a control element disposed in the sensor cartridge, wherein the control element includes all features of the active element with the exception of a molecular receptor such as, for example, an antibody. Other features include an aerodynamic conduit within the device body housing the sensor cartridge. This conduit directs the air onto the sensor element. A dust filter is connected to remove larger undesired particles. A propellor fan draws the air into the device from the surroundings. A microcontroller with or without a liquid crystal display processes the real-time average resistance across the at least one sensor. An internet-of-things (IoT) microprocessor is connected with mobile and web applications enabling users to receive notifications and control the device.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show an exemplary device wherein detection of a target analyte can be viewed via a mobile app: (FIG. 1A) The device mechanism is enabled by: (1) hydrodynamic binding of virus membrane protein with the hygroscopic antibody surface to capture the virus, and (2) conductive network that sensitively responds to viral attachment via change in electron transfer rate. (FIGS. 1B-1C) The prototype design includes a microcontroller with ESP32 internet-of-things (IoT)-circuitry, casing, conduit, and the sensor. (FIG. 1D) The mobile app page designed for communication with ESP32.

FIG. 2 shows an exemplary sensor. Components on the sensor surface include: (1) hygroscopic components for enabling and maintaining protein-protein interaction; (2) conductive nanoparticle and/or polymer components; (3) binding components for preserving the structure of the sensor and prolonged sensor operation and performance; (4) a polymer matrix for controlling electron tunneling; (5) functionalization-assisting molecules for protein attachment; and (6) selective binding agents (antibodies).

FIG. 3A shows preliminary results: % change in resistance of two active devices with respect to control when exposed to aerosolized water and COVID-19 S Proteins. FIG. 3B shows the transduction mechanism of an exemplary device, where the electron tunneling barrier increases with viral attachment, thus increasing the resistance.

FIGS. 4A-4C show CFD simulation of air and viral particles in a room (FIG. 4A) where one person is the source of viral particles, and (FIG. 4B) inside the prototype device chamber. (FIG. 4C) The contour map shows the region on the device element with 35% or more flux of viral particles than the flux at inlet. About 20% of the sensor element has 35% or more flux. Parameters Used: normal temperature and pressure (NTP), volumetric flowrate of air: 0.0017 m3/s. The conduit-walls within the device chamber are ideal reflector.

FIG. 5 shows LEFT: depiction of a viral-particle spike protein and the antibody interaction at the active sensor surface. RIGHT: device specificity as shown; a MERS-CoV spike protein does not bind to the sensor domain.

FIG. 6A shows Raman spectroscopy of the antibody/polyelectrolytic film base sensor (PEDOT-PS5) with characteristic peaks labeled. FIG. 6B shows an optical image (50×) of the scan location of the respective device channel (scale bar is 20 μm). FIG. 6C shows intensity mapping of the C_{α}=C_{β}(—O) bond at ˜1425 cm⁻¹.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In accordance with the principles herein, disclosed are exemplary systems for instantaneous detection of airborne viral particles. Currently, there are no available portable, highly responsive, in-room aerial detection systems for COVID-19 and other airborne viruses that can be easily and widely installed in confined spaces where viral transmission is high.

The disclosed device can provide early warning of aerial presence of contagious viruses and suggest intervention guidelines following a detection event. Once a virus is detected, the users of this technology can wear masks, disinfect their room, open windows, speed up HVAC to increase air circulation, and update vaccination status if needed. In one aspect, the disclosed device can be made available at assisted living facilities, daycares, schools, and hospitals/clinics.

Systems and devices are contemplated herein that can provide a fast and reagent-free detection technology for airborne viruses. Previous attempts at solutions have been lacking due to the required integration of a sensitive detection technology (comparable with PCR tests) with a viral particle capture mechanism (such as gas sensing platforms).

Systems, devices, and methods herein provide laboratory grade detection at a consumer price point (˜$100 for the device and $10 per month for sensor cartridge); continuous and instantaneous detection of a desired airborne pathogen; ease of use by eliminating the dependence on reagents or processing; and reduced device footprint. Systems, methods, and devices contemplated in accordance with the principles herein minimize losses from business interruptions, thus affirming consumer confidence and reducing resource consumption. Systems, devices, and methods herein are not intended to test infection in people after exposure but rather help prevent and/or reduce exposure.

Systems, devices, and methods herein are useful in a wide range of settings, including medical and daycare centers, manufacturing plants, transportation facilities and multi-passenger vehicles such as trains or buses, shopping centers, and a host of additional settings where individuals are subjected to confined spaces with shared airflows. The disclosed systems, devices, and methods allow many businesses to minimize down time, reduce costly over sanitization, and maintain the wellness of their employees and customers.

Systems, devices, and methods herein provide an adaptive, real-time, multi-assay detection platform that can scale to customer requirements.

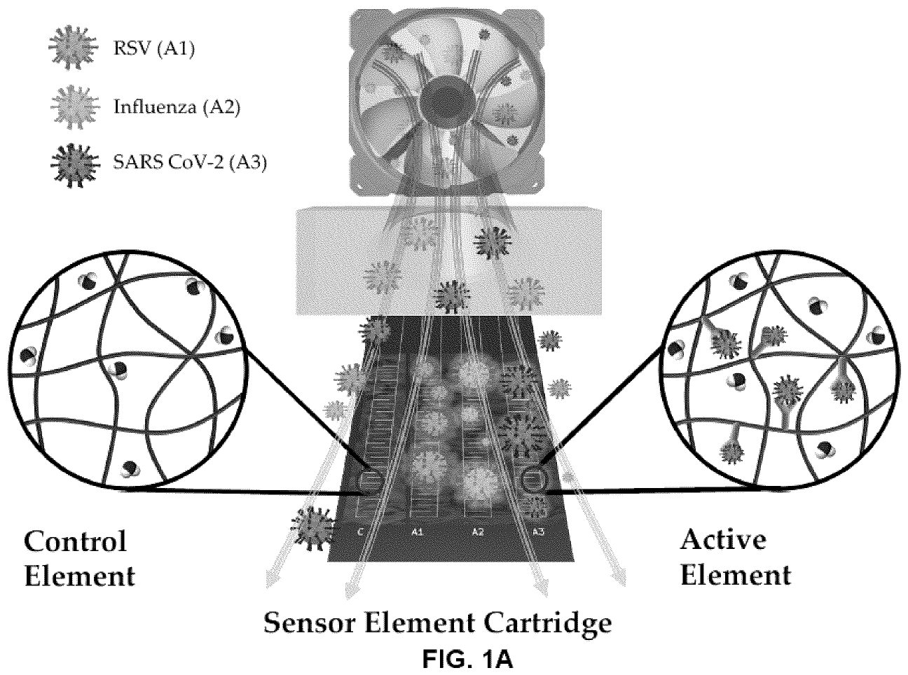

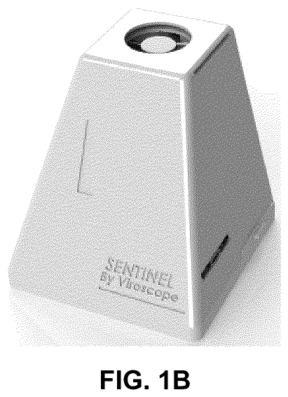

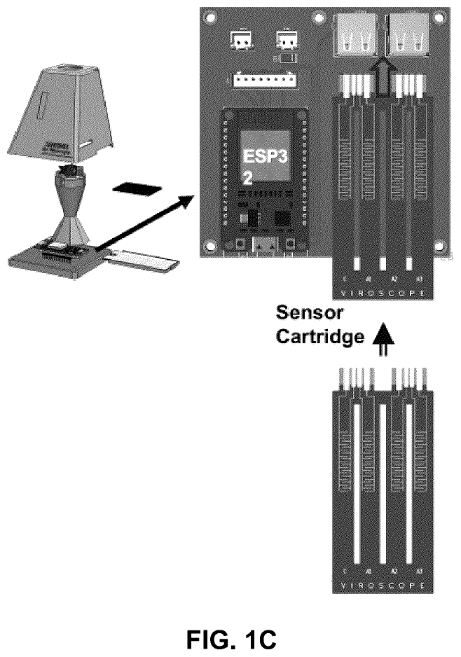

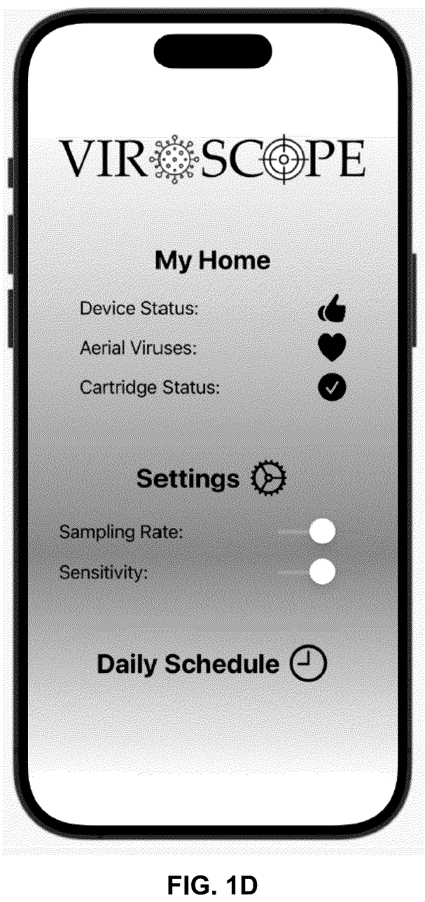

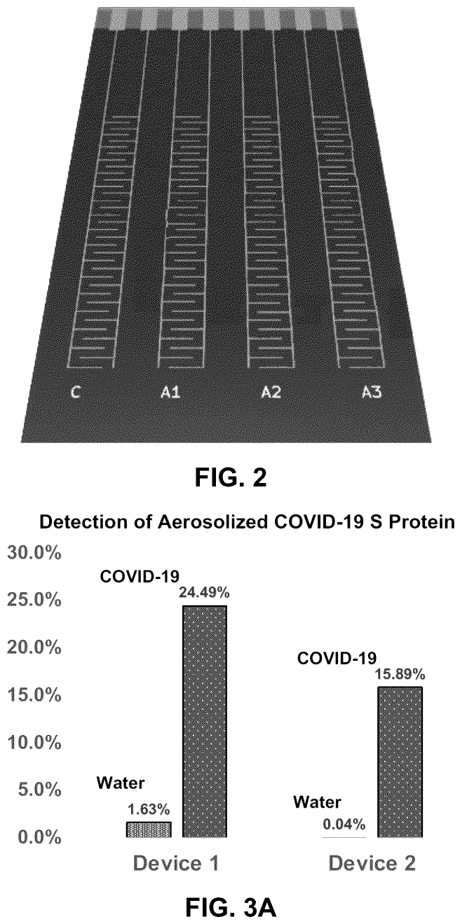

An exemplary device is set forth in FIGS. 1A-1D. In one aspect, the device could be placed in any occupied room, such as in hospitals, homes, hotels, schools, or transport vehicles such as cars, airplanes, trains, cruise ships, and other maritime vehicles, to detect viruses quickly in the immediate surrounding. Exemplary devices and systems herein can achieve detection based on viral concentrations ranging from 0.1×10³ m⁻³ to 40×10³ m⁻³, with a target concentration of about 1-4×10³ m⁻³ in some instances.

Devices and systems herein can be configured as personal devices for travel, can include smart integration with HVAC systems, and can provide go/no-go testing for determining whether individuals wish to enter a given space.

Systems and devices herein can rapidly provide vital environmental health information to the customer about previously undetectable viral contamination in the immediate vicinity so that proper remediation protocols can be observed. Systems and devices herein can provide proactive protection mechanisms in the form of real-time pathogen surveillance that can provide health security for businesses and individuals.

In addition, devices and systems herein can provide important environmental health information in clinical settings and institutions that include hospitals, private clinics, urgent care rooms, and operating rooms, as well as in daycare centers and schools and a number of other indoor settings. Currently, a childcare center that has an exposure will result in staff lost work time and also potentially sick children who must stay home and require supervision, resulting in lost work time for parents as well.

Moreover, clinics and urgent care facilities that have reduced patient turnover and increased wait times due to room sterilization requirements required due to the pandemic, which directly impacts their revenue, can avoid unnecessary sterilization steps based on real-time environmental health information provided by the systems, devices, and methods disclosed herein.

Currently available airborne viral detection methods do not offer real-time detection due to the separate collection and analysis cycles, where the collection of airborne pathogens/particulates occurs at the point of interest, but the analysis of the collected sample occurs at a lab (for example, ThermoFisher “AerosolSense”). Or, if the unit contains both the collection and analysis components (for example Smith Detection “BioFlash”), it is limited by the amount of reagent loaded into the unit, and/or consumes the reagent regardless of the test being positive. This limits how often the unit can perform sample analysis. The first type of instrument is priced around $5,000, and the second type of instrument is priced around $35,000. Both systems require $100/day of PCR reagents, which are consumed for every intermittent measurement (hourly measurement for automated system and daily measurement for external POC), irrespective of the test result (positive or negative), thus consuming costly reagents and producing excessive chemical/biological waste. Further, this significantly delays the onset of detection, which can lead to critical time loss in alerting any occupants of a possible infectious event.

Devices disclosed herein can be or include a portable, fast system that utilizes reagent-free technology for airborne virus detection that can be mass-produced. In one aspect, the device includes a sensor cartridge including at least one sensor, the at least one sensor having a surface with a hydrating component and a conductive component, wherein the conductive component includes an integrated conductive polymer and/or conductive nanoparticles. In a further aspect, the conductive polymer and/or nanoparticles are integrated into the sensor surface. In some aspects, the conductive polymer and/or nanoparticles can be connected to the sensor surface using a binding agent or another means. In another aspect, the hydrating component can be the integrated conductive polymer, an external polyelectrolyte such as, for example, polyallyl hydrochloride, or any combination thereof. Other polyelectrolytes are also contemplated and should also be considered disclosed. In one aspect, in the case of polyallyl hydrochloride, the polyelectrolyte is positive charged and is used in combination with negatively charged conductive polymers and/or nanoparticles.

In one aspect, the integrated conductive polymer can be selected from poly(3,4-ethylenedioxythiophene) polystyrene sulfonate (PEDOT:PSS), polyacetylene (PA), polypyrrole (PPy), poly(p-phenylene) (PPP), or any combination thereof. In some aspects, the device further includes a binding agent, wherein the binding agent binds the integrated conductive polymer to the sensor surface. In one aspect, the binding agent can be polyacrylic acid (PAA). In some aspects, the conductive component is or includes nanoparticles. In a further aspect, the nanoparticles can be silver nanoparticles, gold nanoparticles, or any combination thereof. In a still further aspect, the nanoparticles can be carbon-based nanoparticles such as, for example, carbon dots. In any of these aspects, the nanoparticles can have a size of from about 5 to about 30 nm, or of about 5, 10, 15, 20, 25, or about 30 nm, or a combination of any of the foregoing values, or a range encompassing any of the foregoing values.

In one aspect, and without wishing to be bound by theory, the mechanism of the disclosed device is based on the modification of the electron transport properties (e.g., electron tunneling barrier and carrier doping) in the conducting polymer and/or nanoparticle film via viral protein/antibody binding. In an aspect, the interface of a protein such as, for example, a COVID-19 spike protein, with an antibody for the same increases the potential barrier of the tunneling electrons across the polymer network, directly reducing the probability of tunneling, leading to an observable drop in current.

In some aspects, the sensor cartridge can include an active element including a molecular receptor disposed in the binding agent, conductive component, or hydrating component. In a further aspect, the molecular receptor can be an antibody that binds to at least one viral analyte such as, for example, SARS CoV-2. In one aspect, the SARS CoV-2 (COVID-19) antibody can be selected from one or more of: (1) COV-2 spike RBD antibody (amino acid sequence from Arg 319 to Phe 541; polyclonal rabbit IgG, 40592-T62; Sino Biological, Inc., China), (2) casirivimab and imdevimab, which targets non-overlapping epitopes on the RBD, (3) LY-CoV555, and LY-CoV016 are monoclonal antibodies, which target the S-protein RBD52, and (4) Vir-7831 (Sotrovimab), which is effective on RBD of Omicron variant. In another aspect, the at least one viral analyte can be influenza A53 and the at least one antibody can be selected from one or more of: (1) Influenza A H10 HA Monoclonal Antibody (M001), which binds H10 Hemagglutinin (HA) in several variants, and (2) α2,3-linked sialic acid, which is a receptor responsible for binding with HA, or another antibody (C179, CR6261, Fl6v3). In still another aspect, the at least one viral analyte can be respiratory syncytial virus (RSV), and the at least one antibody can be selected from one or more of: (1) Anti-RSV antibody B1537M, which binds to the F protein, and (2) Anti-RSV glycoprotein antibody 8C5 (9B6), which binds to the G protein. In another aspect, the sensor cartridge can include a control element lacking the molecular receptor but otherwise being identical to the active element.

In another exemplary embodiment, a disclosed detector device can include a base product, or Test-Pod, and a removable/disposable and/or reusable test cartridge can be selectively inserted therein. A cleaning supply can be provided in a kit containing reusable test cartridges. The cleaning supply can be used to clean the device before installing the new cartridge. The test cartridge includes a main COVID/virus sensor element (and, optionally, a control element), which can be used for 30 days, or until a positive detection.

In one aspect, the disclosed devices include a barrier configured to impede conductive electron flow through the devices. In another aspect, the barrier can be or function as one or more of an electron or hole transport barrier, a carrier activation barrier, or a carrier transition barrier. In still another aspect, the barrier is a non-conductive bridge between conductive nanoparticles or conductive polymers. In one aspect, the particular mechanism may be dependent on the final structure of the composite. In a further aspect, the relationship between the change in electron transport or flow rate and virus attachment is similar for all listed mechanisms.

The detector device and/or kit can further include one or more particulate filters. The filter of the base device can be regularly replaced, as recommended, such as about every three months, for example, based on the characteristics of a particular filter material and the environment in which it is located, or through which it passes. In an aspect, the particulate filter or dust filter can be included or connected to the body of the device, wherein the device body also houses the sensor cartridge and, optionally, a fan as well as a liquid crystal display mounted on an exterior of the device body. In one aspect, the dust filter is connected to the system in-line before the fan. Further in this aspect, the air goes through the filter, then through the fan, and then travels to the sensor. In some aspects, the device can further include electrode contacts connected to the sensor cartridge and/or a microcontroller configured to continuously monitor average resistance across the at least one sensor. In some aspects, the electrode contacts are connected to the composite material of the sensor including the conductive polymer or nanoparticles, binding agent, and/or hydrating polymer., and/or to the sensor surface In a further aspect, the electrode contacts can have a gap of from about 100 μm to about 3 mm, or of about 100, 200, 300, 400, 500, 600, 700, 800, or 900 μm, or of 1, 1.5, 2, 2.5, or about 3 mm, or a combination of any of the foregoing values, or a range encompassing any of the foregoing values. In one aspect, a responsive element of the device includes a comparison of a change in resistance at each point of the active element with resistance at each point of the control element. In a still further aspect, the device can include a cooling element operably connected to the sensor cartridge to enhance viral analyte adsorption and a vent disposed in the device body located in a position to increase interaction time within the device body. In one aspect, the device can include an aerodynamic conical conduit in the device body for directing air flow onto the sensor cartridge. In still another aspect, the active element opposes the position of the vent so as to maximize distance between the vent and the active element.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a hygroscopic polymer,” “a virus,” or “an antibody,” include, but are not limited to, mixtures or combinations of two or more such hygroscopic polymers, viruses, or antibodies, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, an “integrated” conductive polymer or an “integrated” nanoparticle refers to a conductive polymer or particle that is integrated into or incorporated into a sensor surface in a disclosed device. The integrated conductive polymer or nanoparticle may further be in contact with a polyelectrolyte, one or more antibodies, or any other component disclosed herein to be on the sensor surface. An integrated conductive material such as a polymer or nanoparticle can be bound to the surface of the sensor using a binding agent or by another means. The integrated conductive material is not a continuous sheet but instead, elements of the integrated conductive material (e.g. polymer chains, nanoparticles, or the like) will have some separation or spacing between individual units in order to enable the electron tunneling or hopping action to occur when the sensor encounters one or more viral particles. In one aspect, the spacing between conductive elements can be on the nanometer scale.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Device Components

Rapid detection of aerial viral particles is a challenge because it requires integration for a sensitive detection mechanism with a hydrated viral capturing surface. The systems and devices disclosed herein can include an antibody-grafted, hygroscopic, and conductive polymer and/or conductive nanoparticle composite that sensitively detects viral particles. Using target-specific antibodies, the systems and devices herein can make use of an electron tunneling or hopping mechanism, enabling a process that can selectively detect airborne COVID-19 virus particles.

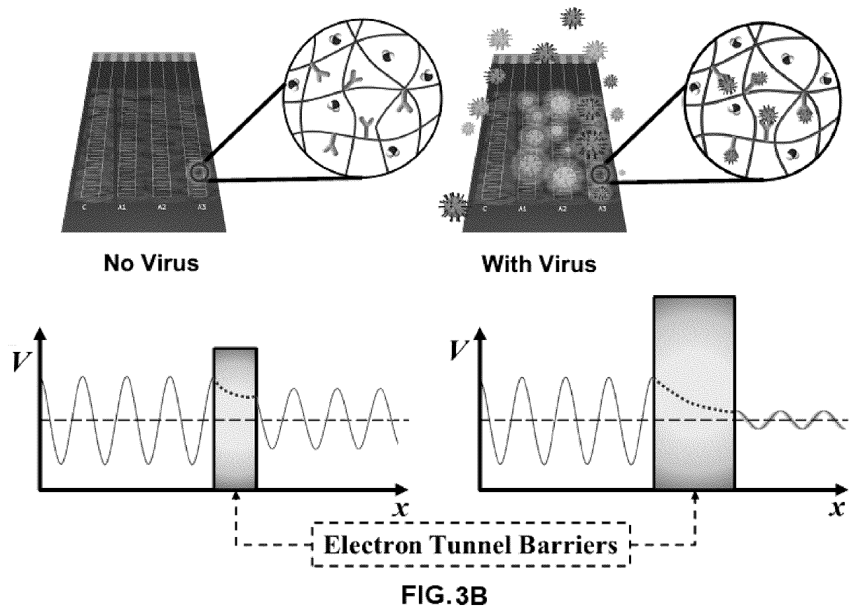

For example, as illustrated in FIG. 3B, an integrated hydrated polyelectrolyte is provided as the base electron tunneling detector, which can be made selective by incorporating spike(S)-protein-binding-antibodies. The device can have a constant bias voltage applied to the detector and the electrical current, via electron tunneling/hopping, across the potential can be monitored in real-time. Upon the attachment of a spike protein of an exemplary virus to an antibody loaded into the detector, a decrease in current (increase in resistivity) will occur, which can trigger a detection alarm when the current passes a certain threshold. The preliminary results show successful base device operation, antibody attachment, and an effective aerodynamic casing.

To establish the sensor operations, several factors must be considered:
(a) Determining the device sensitivity, specificity, and calibration curves for device operation;
(b) developing device positioning guidelines by understanding the temporal behavior of aerial viral particle pathlines in an exemplary room setting/environment from the point of particle origin to the device's air-intake connected sensor-element; and
(c) optimizing the parameters for electron-tunneling and interfacial-interaction between airborne viral particles and self-hydrated composite detector material for rapid aerial-detection technology.

A control sensor-element without the antibodies can be mounted alongside the active sensor (which contains antibodies) to account for local environmental disruptions such as humidity changes, temperature changes, pressure differential from opening doors, and air disturbances from moving room/environment occupants. These sensor elements can be mounted on a substrate to build the cartridge that is loaded into the base device at a specified orientation to ensure proper connection with device electronics. The device electronics can include an Arduino circuit, other suitable circuit, Raspberry Pi, or the like.

The cartridge can be inserted into a plastic housing that can have an inlet fan to force air-convection over the sensor element with strategically placed vents to allow continuous sampling while mitigating backflow. A size selective non-virion filter can be installed to sieve out any foreign material that could potentially interact or deactivate the device (i.e., dust, dirt, and hair).

The sensor element can have a constant bias applied and the resistance can be measured continuously via an electronic circuit configured to monitor the resistance and alarm based on a change in the resistance. For example, in the presence of a viral particle, the spike proteins on the surface of the virus interface with the antibodies on the sensor, leading to a change in the resistivity of the device that triggers a message on the screen and an alarm via the circuit, which can has be programmed based on viral or environmental sensitivity/conditions.

Two principles on which the technology herein, when incorporated into systems and devices herein, works are: (1) molecular interaction leads to a change in electron tunneling between the conductive component's (conductive polymer and/or conductive nanoparticle) units that acts as a sensing transducer, and (2) polyelectrolyte-enabled hygroscopic composite can enable specific protein-protein interaction between viral proteins, such as COVID proteins, and the interfaced antibodies. Here, conductive polymers and/or conductive nanoparticles can be imbedded in hygroscopic polyelectrolyte with functionalized antibodies. Hygroscopic surface(s) can provide wettability in the systems and devices, and interfaced antibodies enable the capture of airborne droplets containing the virus. The combined conductive tunneling and hygroscopicity of the exemplary systems and devices herein can ensure a continuous detection operation.

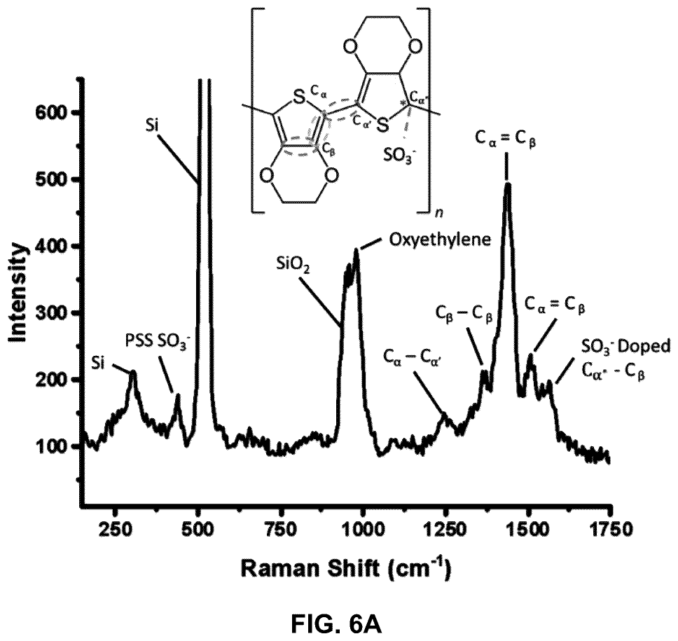

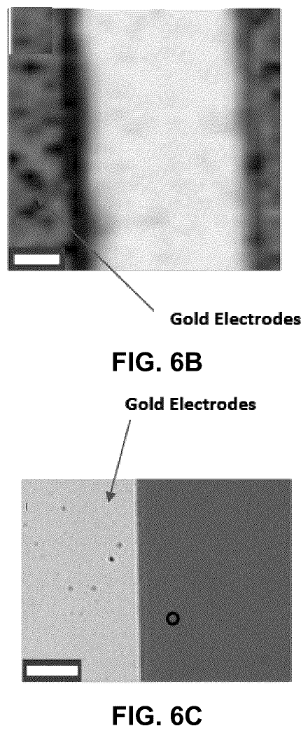

Moreover, exemplary embodiments herein can include a cross-linked hygroscopic polymer/antibody composite structure, which has been studied by Raman spectroscopy as seen in FIGS. 6A-6C. The exemplary device construct with this composite on an electrode device has been probed by IV characterization. To enable aerial interaction with the sensor element, an aerodynamic casing has been designed and developed in accordance with the principles herein; and the flow profiles have been modeled using computational fluid dynamics.

Systems, devices and methods herein deliver quality remote sensing solutions to improve global health and wellness by providing real-time information about the ever-changing environment. Systems, devices and methods herein fundamentally change the approach of airborne contagion detection by capture and simultaneous detection, and enable the proactive identification and monitoring of the first signs of a possible outbreak and help contain the threat before an outbreak can occur.

The COVID-19 virus was used as a model organism to study and develop exemplary devices ad methods for this technology and envision this platform to incorporate other airborne viral contagions leading to multi-assay detection. Products and devices constructed in accordance with the principles herein can provide detection solutions that range from specific localized detection to encompassing passive monitoring devices/connected devices as needed to accommodate a variety of environments, such as aviation manufacturing, airports, train stations and transit hubs, lodging industry, hospitals, schools and daycare centers, and high throughput business such as gyms and restaurants, for example.

Systems and devices herein comprise methodology based on a number of factors, such as device feasibility studies that consider, among other factors, sensitivity, specificity, limit-of-detection, and false positive/negative rate of the aerial detector of a virus, such as COVID-19 for example; detailed aerodynamic calibration curve: device operation and performance with respect to its position/direction in a room setting; and parameter space outline: study the effects of various design parameters (sensor construct/composition, device dimensions/design, fan power) and external parameters (airflow rate/vent-positions, temperature, humidity, and room design).

The results show successful synthesis of self-hydrating, antibody-functionalized, electron-tunneling active composite device. While initial studies were carried out with COVID-S-proteins, systems and devices using complete viral particle droplets for a variety of viruses/virions are contemplated herein as well.

### Example 2: Device Construction and Response Management

The current gap to achieve rapid aerial detection technology is associated with challenges in integrating a self-hydrating surface for viral capture with a sensitive detection mechanism. To address this challenge, devices herein can include a self-hydrating hygroscopic surface conducive to protein-antibody attachment with an integrated conductive polymer network that provides a sensitive detection mechanism via a change in the electron-transport rate (FIG. 3B).

An exemplary device construct (FIGS. 1A-1D) can include:
(a) Sensor element: Each sensor-element (part of a sensor cartridge) can consist of at least two segments: (1) hygroscopic conductive polymer integrated with COVID-19 S-protein antibodies (active device) and (2) hygroscopic conductive polymer without the antibodies (control device). A conductive film will have palladium/gold electrodes to connect the device with suitable internal device circuitry disposed in a base unit.
(b) Electronic circuitry: For the sensor element, a resistor can be added in series to each of the active device and the control device. A constant bias can be applied to the circuit by a suitable microcontroller, such as an Arduino or other microcontroller, which can be programed to measure a voltage drop (Vₜ) every minute (t) across the active device and the control device.
(c) Assembled Device with the Aerodynamic Chamber/Casing: The electronic circuitry can be housed inside a chamber, which can include an aerodynamically designed conical conduit (FIGS. 1A-1D). A slot can be present in the chamber to insert the sensor cartridge to make connection with the internal circuitry, if desired.

Response Measurement: The differential conductivity (V_{D,t}=(Vₜ₊₁−Vₜ)/Vₜ) can be tracked for both the active device and the control device. The change in differential conductivity (□V_{D}=(V_{D,Active,t}−V_{D,Control,t})/V_{D,Control,t}) between the active and control device would imply a difference in the interaction between the control and active device (□V_{D} will be 0 when there is no COVID detected). A change in the conductivity differential will trigger an alert on an integrated display on the device as well as send an automated message to any connected device. Conductivity can be mathematically converted to resistance as needed.

The assembled device can be exposed to (a) aerosolized COVID-19 spike protein, (b) aerosolized MERS spike protein (for control studies), and (c) aerosolized blank sample in air via nebulizer. The change in the differential conductivity (□V_{D}) as a function of concentrations of proteins in air (calculated from the solution concentration) can then be determined. The following are the specifications:

Calibration Curve: The analysis of the data from the response measurement leads to a calibration curve: Aerial concentration (C) as a function of the differential conductivity (C=f(□V_{D})).

Limit of Detection (LOD): The calibration curve and the standard deviation in □V_{D} measurements (SD) for a blank sample and low-concentration sample can be evaluated to find the LOD=LOB+1.645 (SD_{Low-Concentration}); where LOB (limit of black)=mean_{Blank}+1.645 (SD_{Blank}). Target LOD<2000 m⁻³.

Sensitivity and Specificity: can be evaluated by extensive tests with COVID and MERS proteins to determine the false positives and negatives. Sensitivity=(True Positives (A))/(True Positives (A)+False Negatives (C)); Specificity=(True Negatives (D))/(True Negatives (D)+False Positives (B)). To determine the binding specificity, control studies can be conducted using a spike protein from a similar, but different strain of beta corona virus family, MERS-CoV (MERS-CoV spike protein 1 Met to 725 Glu (from mybiosource, catalog number MBS434229).

Operating conditions: The sensitivity and specificity studies can also be conducted at different levels of humidity and temperature to determine the operational range of the device.

### Example 3: Device Mechanism and Preliminary Results

The central mechanism of the aerial COVID sensor device is based on the modification of the electron-transport properties (electron tunneling barrier and carrier doping) in the conducting polymer film via COVID-S-protein/antibody binding (FIG. 5). Here, the conducting network is that of a conductive polymer (e.g. PEDOT:PSS, PA, PPy, PPP); and the hydrating component can be either an external polyelectrolyte (polyallyl hydrochloride) or the conductive polymer itself. Other suitable conducting polymers and hydrating components are contemplated as well.

Electron Transport Mechanism: When the exemplary sensor-element is placed in an electric field via connected electrodes, electrons transport from one polymer unit to the next by tunneling or hopping through molecular junctions/connections between the units. The electronic structure of these junctions governs the electron transport across the junctions, while the density of states in the polymer units governs its charge-carrier density. This electron-transport through the junction can occur via one of the following mechanisms: direct-tunneling, Fowler-Nordheim tunneling, electron hopping (e.g., variable range hopping), or thermionic emission. Studies conducted with conductive polymers (PEDOT:PSS) have shown that the electron transport mode is variable range hopping. Simmon's model for this electron-tunneling process through molecular junction is given by:

$$J = / \frac{e}{2 π {hd}^{2}} \{ ( {φ}_{B} - \frac{eV}{2} ) exp [ - \frac{4 π {d ( 2 m )}^{\frac{1}{2}}}{h} {( {φ}_{B} - \frac{eV}{2} )}^{\frac{1}{2}} ] - ( {φ}_{B} + \frac{eV}{2} ) exp [ - \frac{4 π {d ( 2 m )}^{\frac{1}{2}}}{h} {( {φ}_{B} + \frac{eV}{2} )}^{\frac{1}{2}} ] \}$$

where J is the current density, Vis electron potential, ϕ_{B} is the tunneling barrier that is modified by protein/antibody interaction, d is the electron tunneling distance, m is the electron mass, e is the electronic charge, h is the Planck's constant, and a is an adjustment factor for the shape of the barrier and for the effective mass of electron (α=1 for rectangular barrier and ˜0.65 for organics). When the energy difference between nearest neighboring sites is large, the electron tunneling probability will be small. In this case, the electrons hop to a site further along with smaller energy difference, and the phenomena is termed variable range hopping (VRH) mechanism. In this case, the conductivity (σ) is expressed as:

$$σ = {σ}_{0} ( T ) {exp ( \frac{{T}_{0}}{T} )}^{β}$$

where σ₀ is the temperature dependent prefactor (σ₀ (T)=AT^{m}) (T₀ is the characteristic temperature and β is the characteristic exponent related to the dimensionality of the system (β=¼).

The sensing element can include a suitable PEDOT:PSS scaffold functionalized with viral antibodies, such as COVID S-protein antibodies, for example. The schematic also shows the electron tunneling mechanism, where electrons transport from the negative terminal to the positive terminal by hopping through the molecular junctions when under electric field.

Protein Attachment Confirmation: For each of device, it is important to confirm protein attachment. For example, there are four major proteins encoded by the COVID-19 genome: the spike(S) protein, nucleocapsid (N) protein, membrane (M) protein, and the envelope (E) protein. Due to its location on the outer shell of COVID-19 and its functional importance, the S-protein's specific binding with antibodies (integrated into a network of conductive-polymers) is discussed herein. Structurally, the 1270-90 amino acid S-protein is a heavily glycolated type-I membrane protein composed of a transmembrane anchor, and a large ectodomain, which includes the receptor-binding domain (RBD) (S1). This S1 segment of the protein is responsible for binding with the host cell receptors (such as angiotensin converting enzyme 2 (ACE-2)), which mediates the entry of the virus into the target cells.

The RBD segment of the recombinant S-protein of human coronavirus (SEQ ID NO. 1) can be employed for binding studies, for example. The protein is produced with mammalian proteins (particularly, Chinese hamster ovary cells). The purity is more than 90%, which was tested by Sodium Dodecyl Sulfate Polyacrylamide Gel Electrophoresis (SDS-PAGE) analysis. The primary amino acid sequence of this SARS-CoV-2 spike protein 319 Arg to 541 Phe (from Ab cam, catalog number ab27065) with a molecular weight of 39 kDa (FIG. 5). The PEDOT:PSS base sensor has been probed via Raman spectroscopy to observe a uniform deposition of the film on the sensor surface (FIG. 6A).

Further, identified herein is the C_—Cᵢ, C_═C_and C_—Cᵢ peaks, representing the vibrations of the corresponding bonds in the crosslinked PEDOT-PSS film with attached COV-2 spike RBD antibody (amino acid sequence from Arg 319 to Phe 541; polyclonal rabbit IgG, 40592-T62; Sino Biological, Inc., China) (FIG. 6A).

Binding Kinetics: The binding studies can be conducted on the conductive polymer device (functionalized with antibodies) exposed to aerosolized S-protein (in air) in a chamber. These surfaces can be tested with fluorescence microscopy and Raman spectroscopy. The measurements can also be correlated with the corresponding electrical studies performed in parallel.

Specifically, the studies can be conducted by changing the concentration of the aerosolized S-protein (1 ppm-1000 ppm), the density of antibodies in the composite (also measured by Raman spectroscopy), composite temperature (0-35° C.), and aerial humidity (10-80%); while simultaneously acquiring the Raman spectra and the electrical conductivity data. These data sets can also be acquired for control devices: (a) no antibodies in the composites, (b) mismatched antibodies in the composite, and (c) mismatched aerosolized protein (in air). Further, the confocal fluorescence microscopy can provide confirmation on the expected binding of the tagged antibodies polyelectrolytic/conductive-polymer backbone. These studies can be used for the development of the calibration curve that will guide the aerial detection of the viruses.

### Example 4: Aerodynamic Modeling of COVID Particle Pathlines

The flow dynamics and aerosolization of respiratory COVID-19 droplets has been a critical aspect of the spread of its infection. The airborne droplets originate from the respiratory tract of the infected person during coughing, sneezing, talking, and breathing.

Once the particles are airborne, the effective forces on the droplet include inertia, gravity, evaporation, and convection (surrounding airflow). Here, the smaller particles evaporate faster than they settle and stay airborne as a virus nuclei. Further, the viral load droplets, their number density, and size distribution play a critical role in their flow dynamics.

Computational fluid dynamics (CFD) studies can be performed to understand the transport mechanics of the airborne viral droplets under convective force (an air fan) to determine the interaction and transferability of the particles to the sensor element. The model correlates the particle dynamics with the fan power, device geometry, droplet size, viral-load, and distance/location of origin. ANSYS CFD software can be used for these studies. Briefly, the governing equations include conservation laws for mass, angular momentum, energy in the Cartesian coordinate system, and particle absorption/accretion on the active sensor element. These can be written as follows:

$$\frac{δρ}{δ t} + \frac{δ ( ρ {u}_{i} )}{δ {x}_{i}} = 0 ;$$

$$\frac{δ ( ρ {u}_{i} )}{δ t} + \frac{δ}{δ {x}_{j}} ( ρ {u}_{i} {u}_{j} ) + \frac{{δ}_{P}}{δ {x}_{i}} = \frac{δ}{δ {x}_{j}} ( {τ}_{ij} + {τ}_{ij}^{R} ) + {S}_{i} ;$$

$$\frac{δρ H}{δ t} + \frac{δ}{δ {x}_{j}} ( ρ {u}_{i} H ) = \frac{δ}{δ {x}_{j}} ( {u}_{j} ( {τ}_{ij} + {τ}_{ij}^{R} ) + {q}_{i} ) + \frac{δ p}{δ t} - {τ}_{ij}^{R} \frac{δ {u}_{i}}{δ {x}_{j}} + ρε + {S}_{i} {u}_{i} + {Q}_{H} ,$$

where H=h+ (u²/2), u is the fluid velocity, ρ is the fluid density, Sᵢ is a mass-distributed external force per unit mass due to a porous media resistance (Sᵢ^{porous}), a buoyancy (Sᵢ^{gravity})=−ρgᵢ, where gᵢ is the gravitational acceleration component along the i-th coordinate direction), and the coordinate system's rotation (Sᵢ^{rotation}), i.e., Sᵢ=Sᵢ^{porous}+Sᵢ^{gravity}+Sᵢ^{rotation}, h is the thermal enthalpy, Q_{H} is a heat source or sink per unit volume, Tᵢₖ is the viscous shear stress tensor, qᵢ is the diffusive heat flux.

The subscripts are used to denote summation over the three coordinate directions. For Newtonian fluids, the viscous shear stress tensor is defined as

$${τ}_{ij} = μ ( \frac{δ {u}_{i}}{δ {x}_{j}} + \frac{δ {u}_{j}}{δ {x}_{i}} - \frac{2}{3} {δ}_{ij} \frac{δ {u}_{k}}{δ {x}_{k}} ) .$$

Here, the particle's aerial density (mass/aerial-volume) is assumed to be 0-5% of the air density. Further, the interaction of the particle with the active sensor surface can be accounted for by absorption/accretion parameters.

### Example 5: Aerodynamics Inside the Device Chamber

The main objective of the CFD studies is to understand the droplet aerodynamics within the exemplary device chamber/conduit to maximize the interaction between the COVID droplets entering the device and the sensor element of the device. One exemplary conduit design has a conical shape that utilizes the flow dynamics of a throttle valve by directing the flow into a small area where the sensor element is placed. This design with vents expedites droplet sampling and maximizes the droplet residence time. CFD modeling can be carried out by varying the droplet size, flow-rate, density, fan parameters (dimensions, power, direction, continuous and pulsed), and interaction parameters. Droplet/sensor interaction efficiency can be studied to guide (a) conduit shape/size, (b) chamber dimensions/shape, (c) vent positioning, and (d) sensor placement. These studies can be complemented with experimental studies with 3D printing chamber/conduit (FIGS. 1A-1D) of exemplary designs tested with solution droplets containing SARS-CoV-2 spike proteins according to actual observed patient viral loads.

### Example 6: Detector Design and Optimization of Operation

The data on feasibility tests, aerodynamics, interfacial-interactions, and electrical properties can guide device design parameters for exemplary devices and systems constructed in accordance with the principles of the present disclosure. Further aerodynamic studies can guide the optimization of device placement in a typical room or other structure setting.

### Parameter Space

Each sensor-element cartridge can include an active device (with antibodies) and a control device (without any antibody). The parameters that can be considered include surface area of the device (active and control), gap between the interdigitated electrodes, and conductive material-to-antibody ratio on the active device.

The aerodynamic and interfacial studies can guide the following: placement of the sensor with respect to the conduit exit, size of the conduit, size/power of the fan, and exit vent placement. The goal of these design parameters will be to increase the residence-time and interaction efficiency of the incoming droplets to maximize particle capture.

Electronic Circuit: Currently, an Arduino microcontroller or other suitable controller can be used to read the voltage drop across the sensor element and to process the data to determine ΔV_{D} and output the results (Detected or Not Detected). Based on the non-linearity of the calibration curve, the microcontroller can be programmed to maximize sensitivity. Further, Arduino-wifi or other suitable wired or wireless connection can be used to transmit the results to a connected device.

### Example 7: Room Aerodynamics for Device Positioning

To understand the dynamics of particle flow pattern in a room from origin (such as an infected person) to the sensor element (within in the main device) given room air flow parameters can inform: air-flow supply, vents' location, origin of infection, device placement/orientation, room size, internal architecture, and obstructions (furniture etc.). This information can be used to build a set of guidelines for optimized placement of the base sensor in different room settings. The results obtained can be used as feedback to the sensor design.

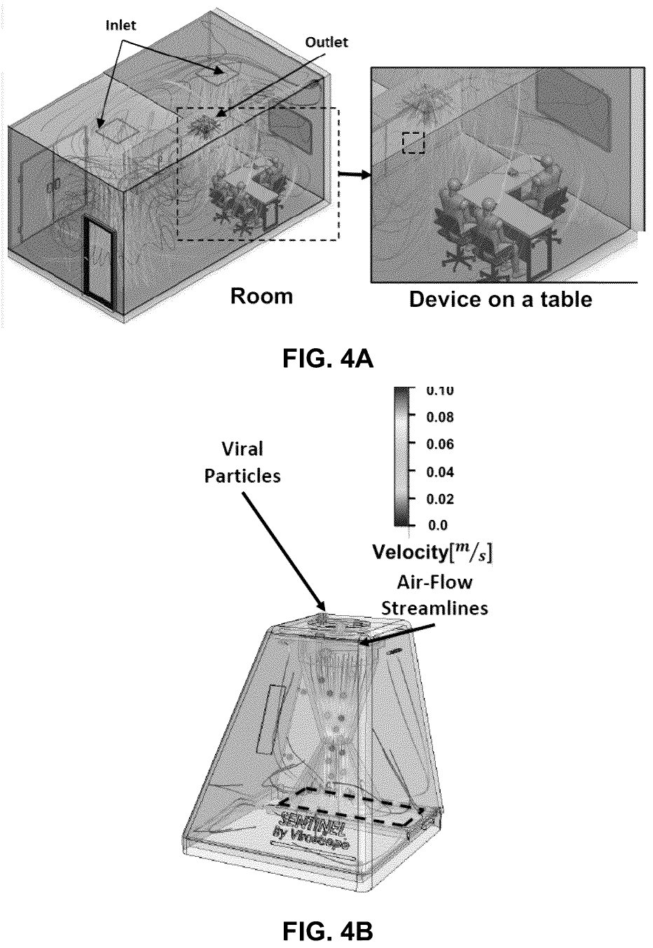

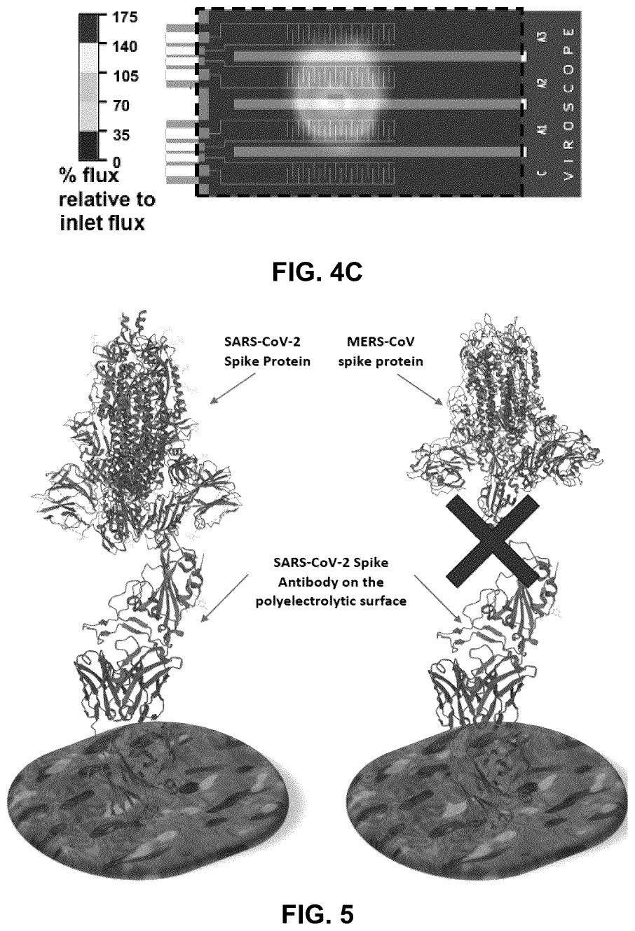

For example, the CFD models will provide information on how the simulated streamlines of the COVID droplets are affected by the device's fan specifications (size, power, continuous or pulsed); and how the particle's interaction with the device element is influenced by droplet size, viral-load, and device location/orientation. Results of one such study on an office space with an exemplary device on a table is shown in FIGS. 4A-4C. The optimum position of the detection device with an air intake of 3.5 cfm is on the work desk according to the HVAC simulation study for an 18×10^{×7.5} ft office space. This position allows optimal air sampling in the enclosed space while ensuring the COVID particle-base device interaction.

A well-designed, widely implementable airborne pathogen detection system that can be integrated within a room setting to provide accurate information about the presence of infectious aerial particles has the potential to transform humans' ability to make decisions about safe usage of space. Currently available aerial detection systems are bulky, require capture of the particles, and require sample processing (automated or at remote location) in liquid media. Clearly, their size makes it challenging to integrate into room settings and their processing time increases the probability of occupant-exposure. Systems constructed in accordance with the principles herein will allow instant detection results and will have a small footprint (4-6 square-inches).

Further, the absence of information on viral presence in the surrounding air has led to the implementation of exposure minimization procedures, such as mandates on face-masking, social distancing practices, work from home policies, and occupancy limitations. An aerial-surveillance can be an effective first level of the exposure-mitigation strategy to guide the next levels, such as masking and social-distancing, or even isolation/disinfection of a room. It can also provide targeted information on localization or scale of potential exposure. Integrating IoT protocols into the device will provide proactive decision making by providing information about the presence of a contagion in regularly frequented places.

The COVID-pandemic had a major impact on the economy due to shut-downs with widespread fear for life and health, arising from not knowing if the air has infectious viral particles. Towards economic benefits, portable aerial detection devices constructed in accordance with the principles herein will (1) provide safe environment for schools, businesses and hospitals improving system efficiencies, (2) reduce testing costs and wait-times, and (3) help preserve precious PPE resources. Further, an active and seamless airborne monitoring system will enable prevention of widespread transmission event due to the rapid detection and notification. Reducing the spread of the contagion reduces the number of infected persons and minimizes the loss of productivity. Integration of systems and devices constructed in accordance with the principles herein and installed in workplaces can help maintain team productivity, reduce over sanitation, prevent needless testing, and reduce the healthcare cost, especially for small businesses.

In accordance with the principles herein, a versatile detection platform technology can be achieved for other or additional infectious bio-agents. An exemplary hydroscopic polymer-nanoparticle matrix system herein can operate on the principle of electron hopping mechanism to detect viral particles at high sensitivity. Using target-specific antibodies, an exemplary process that can selectively detect airborne COVID-19 virus particles and/or other infectious bio-agents was developed herein. False positive/negative results can also be addressed in accordance with the principles herein. While the underlying technology is highly sensitive, there can be false positive or negative results due to non-specific binding. This affect can be mitigated by providing non-specific binding polymers such as polyethylene glycol, for example.

### Example 8: Multi-Virus Detecting Device

The disclosed multi-virus detecting device includes a soft, self-hydrating hygroscopic surface and an integrated percolating network of conductive polymer with electron-tunneling junctions. The disclosed device leverages the capability of the hygroscopic surface to facilitate protein-antibody binding, while a near-percolating network (˜8-15%) of conductive polymer provides a highly sensitive mechanism for detecting aerial particles via changes in the electron-transport barrier (FIG. 3B). This integrated approach offers an efficient solution for the rapid detection of airborne viral particles. The technology introduces a novel composite design of conductive-polymer: filler/binder-antibody on hygroscopic substrate offering robust electron tunneling sensitivity while enabling protein binding; and is not based on electrochemical conductive polymers electrodes, which are not stable due to polymer leaching in solution.

The disclosed device design incorporates a unique approach that involves the grafting of target-specific antibodies (such as S-protein antibodies for COVID-19, anti-hemagglutinin protein/sialoglycoprotein for influenza-A H1N1/H3N2, and Anti-RSV gpF for RSV) onto a polymer composite consisting of three components: (1) a hygroscopic component that offers localized hygroscopicity to facilitate the binding of virus's membrane protein, (2) a percolating network of conductive polymer that provides a sensitive electron-tunneling-based transduction mechanism for selective detection of the virus, and (3) a binding and filler polymer as tunneling junctions where antibodies are bound. This approach ensures a highly sensitive and specific detection of the target viruses, with minimal interference from other airborne particles or contaminants.

The overall device architecture, as shown in FIGS. 1A-1C, has three segments: (i) Sensor Cartridge: This cartridge will have three active sensor elements: COVID-19, Flu (H1N1, H3N2), RSV, and one control element, all on one printed circuit board (PCB). The elements include hygroscopic polymer, conductive polymer, and binding agents. The active elements contain specific antibodies: S-protein antibodies (COVID-19), anti-hemagglutinin protein/sialoglycoprotein (Flu—H1N1, H3N2), and Anti-RSV gpF (RSV). The sensor cartridge connects to a receiver, which is electrically connected to the base unit's circuitry. (ii) Data Acquisition and Signal Processing: A microcontroller (Arduino) has been programmed to continuously measure the 30-second average resistance across active and control sensor elements. The microcontroller constantly compares the change in resistance of the active device with respect to the control every second to reduce the possibility of false-positives or negatives. (iii) Aerodynamic Chamber/Casing: The device's casing will include an aerodynamically designed conical conduit with a filter to remove big particles and a small propeller fan (FIGS. 1A-1C) that will direct and concentrate airborne viruses towards the sensor element.

Electron Transport Transduction: Under an electric field, electrons travel from one polymer unit to another through filler-polymer junctions. The tunneling barrier of these junctions (FIGS. 3A-3B) have exponential relation to the electron transport rate. This electron transport can occur through various mechanisms, such as direct-tunneling, Fowler-Nordheim tunneling, electron hopping (e.g., variable range hopping), or thermionic emission. When an exterior protein of the virus attaches to the antibody on the device, it causes an increase in electron tunneling/hopping barrier, which decreases the electric current (increases resistivity), which is registered as a detection event by the microprocessor. FIGS. 3A-3B demonstrate successful airborne detection of COVID protein.

Sensor Element Construct: The central principles behind the technology are: (1) sensitive transduction via modification of electron tunneling barrier, and (2) soft, hygroscopic composite enable protein binding between virus' membrane-proteins and complementary antibodies. Here, the polymers composite are formed with the conductive polymer (PEDOT:PSS, polyaniline, or polypyrrole (PPy)) mixed with binding/filler agent (85-92%) spin-coated on a layer of crosslinked hygroscopic polyelectrolyte (such as poly-allylamine hydrochloride), followed by antibody functionalization via amide bonding chemistry (EDC-NHS) with the binder. The hygroscopic surface provides wettability for molecular interaction, and interfaced antibodies enable the capture of airborne droplets containing the virus. The device functions as a percolating network of electron-tunneling junctions that can sensitively detect specific protein-protein binding.

Preliminary Data: Two active devices, which were functionalized with COVID-19 S-RBD-binding antibodies, and a control device (without antibodies) were tested for their response to aerosolized COVID S-proteins (FIGS. 3A-3B). The antibody attachment was confirmed by Raman spectroscopy. The aerosolization parameters were set to 7×10⁴ droplets/m³, with a size of approximately 2-5 μm, and an estimated aerial density of 1.23×10⁵ proteins per m³. Since each viral particle contains about 30 trimeric S-glycoproteins, implying ˜90 RBD-S proteins, this concentration is equivalent to ˜1.36×10³ aerial viral particles per m³, which is in the range of the clinical aerial concentration found near infected patients, and can be considered the concentration at which infection might occur. Upon exposure to the COVID-19 S-protein aerosol, the two devices showed an increase in resistance by 24.49% and 15.89%, respectively, with respect to the control device, following ˜2-5 seconds of aerial exposure, and a 6-minute relaxation time. This constitutes clear proof-of-concept for specific and rapid aerial detection. Conversely, an insignificant change in resistance (1.63% and 0.04%) was observed in response to aerosolized de-ionized water (blank sample). The difference in the response of the two devices is attributed to the potential differences in the entering concentration of COVID-19 S-protein delivered from the nebulizer. Stability: The stability of the device was tested with 300 cycles of 10 s nebulized water exposures. The maximum change in resistance with respect to control device is less than 1% (average ˜0.35%).

Several active sensor elements can be developed from the following antibodies for specific attachment of viruses:

For COVID-19 antibodies: (1) COV-2 spike RBD antibody (amino acid sequence from Arg 319 to Phe 541; polyclonal rabbit IgG, 40592-T62; Sino Biological, Inc., China), (2) casirivimab and imdevimab, which targets non-overlapping epitopes on the RBD, (3) LY-CoV555, and LY-CoV016 are monoclonal antibodies, which target the S-protein RBD52, and (4) Vir-7831 (Sotrovimab), which is effective on RBD of Omicron variant.

For Influenza A53: (1) Influenza A H10 HA Monoclonal Antibody (M001), which binds H10 Hemagglutinin (HA) in several variants, and (2) α2,3-linked sialic acid, which is a receptor responsible for binding with HA. There are several other antibodies (C179, CR6261, Fl6v3) that will be explored if these two fail.

For RSV, both G and F proteins will be targeted: (1) Anti-RSV antibody B1537M, which binds to the F protein, and (2) Anti-RSV glycoprotein antibody 8C5 (9B6), which binds to the G protein.

Preliminary Results: FIGS. 4A-4C depict a computational fluid dynamics (CFD) simulation of the streamlines of air and viral particles as they travel in a room from an infected person into the device's conical conduit and towards the sensor element, under the influence of convective forces generated by a propeller fan (1 inch diameter and 3.5 ft³ per minute air flow rate). The device casing includes conical conduit designed to concentrate the viral flux by two folds and vents to enable a constant flow of air for sampling purposes. The device casing includes vents to enable a constant flow of air for sampling purposes. In FIG. 4C, a contour map represents the interaction of the virus with the surface of the sensor element. Here, the region with viral flux higher than 35% of inlet flux (at the fan) is shown. Currently, this area is about 20% of the sensor element (and can be increased to 60% by modifying chamber design). For this CFD72 simulation, the office space measures 18×10^{×7.5} ft³ with standard heating, ventilation, and air conditioning (HVAC) setup. The simulated room has a total of three individuals with one of them infected and exhaling viruses while speaking. The positioning of the device was optimized to allow for optimal air sampling within the enclosed space, directing as many virus particles as possible towards the base device. The results of the simulation indicated that 10% of the virus particles entered the detector due to the convective force of the inlet fan. This implies that about 83 to 250 particles enter the device per minute if there is one infected person talking in a 180 square foot room depending on where the device is placed with respect to the person (4-10 ft away from the device) (FIG. 4A). This number is greater than 500 for coughing, sneezing or severe infections if the device is 4-10 ft from the infected person. Further, a fraction of these particles entering the device will get attached to the sensor element.

Design the Chamber to Improve Air Sampling and Viral Interaction: The detection limit of the device can be improved by increasing particle interaction with the sensor. To achieve this, the computational model can analyze interaction contour maps, as shown in FIG. 4C, to determine the percentage area of interaction for different design parameters. These parameters include the geometry and orientation of the conduit outlet, the placement of the sensor cartridge in the detection chamber, and the geometry and position of airflow traps, baffles, and vents. Additionally, the airflow rate within the device will be adjusted by varying fan specifications, such as flow rate and operation mode. Further, strategies to concentrate the viral concentration will include reducing the outlet area of the conical conduit to increase the viral flux and studying the position and number of the fan inlets to make the detection effective. Once the computational model predicts at least 60% of sensor elements has high viral flux, the device will be fabricated via 3D printing for testing.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Ashmawy, N. H. et al. Novel carbon/PEDOT/PSS-based screen-printed biosensors for acetylcholine neurotransmitter and acetylcholinesterase detection in human serum. Molecules 24, (2019).
2. Behura, S. K., et al. Graphene-semiconductor heterojunction sheds light on emerging photovoltaics. Nat Photonics 13, 312-318 (2019).
3. Bergeron, S., et al. Evaluating mixtures of 14 hygroscopic additives to improve antibody microarray performance. Anal Bioanal Chem 407, (2015).
4. Berry, V. et al. Modulation of electron tunneling in a nanoparticle array by sound waves: An avenue to high-speed, high-sensitivity sensors. Small 7, (2011).
5. Bhardwaj, S. K. et al. Recent progress in nanomaterial-based sensing of airborne viral and bacterial pathogens. Environ Int 146, 106183 (2021).
6. Bourouiba, L. Turbulent Gas Clouds and Respiratory Pathogen Emissions. JAMA (2020) doi: 10.1001/jama.2020.4756.
7. Bueno de Mesquita, P. J., et al. Control of airborne infectious disease in buildings: Evidence and research priorities. Indoor Air vol. 32 Preprint doi: 10.1111/ina. 12965 (2022).
8. Carvalho Fernandes, D. C., et al. 3D-printed graphene/polymer structures for electron-tunneling based devices. Sci Rep 10, 1-8 (2020).
9. Casanova, L. M., et al. Effects of air temperature and relative humidity on coronavirus survival on surfaces. Appl Environ Microbiol 76, (2010).
10. Cesewski, E. et al. Electrochemical biosensors for pathogen detection. Biosens Bioelectron 159, 112214 (2020).
11. Chang, Y. S. et al. Development of Fluorescence-Tagged SARS-CoV-2 Virus-like Particles by a Tri-Cistronic Vector Expression System for Investigating the Cellular Entry of SARS-CoV-2. Viruses 14, (2022).
12. Che, S. et al. Retained Carrier-Mobility and Enhanced Plasmonic-Photovoltaics of Graphene via ring-centered η6Functionalization and Nanointerfacing. Nano Lett 17, 4381-4389 (2017).
13. Chia, P. Y. et al. Detection of air and surface contamination by SARS-CoV-2 in hospital rooms of infected patients. Nat Commun 11, 2800 (2020).
14. Cortese, M. et al. Advanced microscopy technologies enable rapid response to SARS-CoV-2 pandemic. Cellular Microbiology vol. 23 Preprint doi: 10.1111/cmi. 13319 (2021).
15. Davis, H. E., et al. Long COVID: major findings, mechanisms and recommendations. Nature Reviews Microbiology vol. 21 Preprint doi: 10.1038/s41579-022-00846-2 (2023).
16. de Mesquita, J. B. et al. Estimating exposure to infectious influenza aerosols in roommates of influenza cases during the 2012-2013 season on the University of Maryland campus. in 15th Conference of the International Society of Indoor Air Quality and Climate, INDOOR AIR 2018 (2018).
17. Debbarma, R., et al. Defect guided conduction in graphene-derivatives and MoS2: Two-dimensional nanomaterial models. Appl Mater Today 23, 101072 (2021).
18. Debbarma, R., et al. WS2-induced enhanced optical absorption and efficiency in graphene/silicon heterojunction photovoltaic cells. Nanoscale 10, 20218-20225 (2018).
19. Deng, S. et al. Graphene Wrinkles Enable Spatially Defined Chemistry. Nano Lett 19, 5640-5646 (2019).
20. Deng, S., et al. Strain in a single wrinkle on an MoS 2 flake for in-plane realignment of band structure for enhanced photo-response. Nanoscale 11, 504-511 (2019).
21. Domingo, J. L., et al. Influence of airborne transmission of SARS-CoV-2 on COVID-19 pandemic. A review. Environ Res 188, 109861 (2020).
22. Domingo, M. et al. Interaction between SARS-CoV-2 spike glycoprotein and human skin models: a molecular dynamics study. Soft Matter 17, (2021).
23. Edwards, D. A. et al. Exhaled aerosol increases with COVID-19 infection, age, and obesity. Proc Natl Acad Sci USA 118, (2021).
24. Fan, X. et al. PEDOT:PSS for Flexible and Stretchable Electronics: Modifications, Strategies, and Applications. Advanced Science 6, (2019).
25. Fenoy, G. E., et al. Functionalization strategies of PEDOT and PEDOT:PSS films for organic bioelectronics vol. applications. Chemosensors 9 Preprint doi: 10.3390/chemosensors9080212 (2021).
26. Fischer, M. J. E. Amine coupling through EDC/NHS: a practical approach. Methods Mol Biol 627, (2010).
27. Fraser, C., et al. Factors that make an infectious disease outbreak controllable. Proc Natl Acad Sci USA 101, (2004).
28. Guziejko, K., et al. Long COVID. Przegl Epidemiol 76, (2022).
29. Hardi, G. W. et al. Enhancing the sensitivity of dopamine biosensor using PEDOT-PSS modified glassy carbon electrode. in AIP Conference Proceedings vol. 2344 (2021).
30. Ho, M. S. et al. Preparing to prevent severe acute respiratory syndrome and other respiratory infections. Lancet Infectious Diseases vol. 4 Preprint doi: 10.1016/S1473-3099 (04) 01174-0 (2004).
31. Hu, Y. F. et al. In-Silico Analysis of Monoclonal Antibodies against SARS-CoV-2 Omicron. Viruses 14, (2022).
32. Hummelgen, I. A., et al. Polymer and polymer/metal interface characterization via Fowler-Nordheim tunneling measurements. Appl Phys Lett 68, 3194-3196 (1996).
33. Jain, D., et al. Role of Hygroscopic Low Molecular Mass Compounds in Humidity Responsive Adhesion of Spider's Capture Silk. Biomacromolecules 19, (2018).
34. Jasuja, K. et al. Introduction of Protonated Sites on Exfoliated, Large-Area Sheets of Hexagonal Boron Nitride. ACS Nano 12, 9931-9939 (2018).
35. Jasuja, K., et al. Reversibly compressible and stretchable ‘springlike’ polymeric nanojunctions between metal nanoparticles. Small 4, (2008).
36. Ji, T. et al. Detection of COVID-19: A review of the current literature and future perspectives. Biosens Bioelectron 166, 112455 (2020).
37. Johnson, M. Antibody Storage and Antibody Shelf Life. MATER METHODS 2, (2012).
38. Jones, B. E. et al. The neutralizing antibody, LY-CoV555, protects against SARS-CoV-2 infection in nonhuman primates. Sci Transl Med 13, (2021).
39. Ke, Z. et al. Structures and distributions of SARS-CoV-2 spike proteins on intact virions. Nature 588, (2020).
40. Kim, S., et al. Cellular Nano-Transistor: An Electronic-Interface between Nanoscale Semiconductors and Biological Cells. Mater Today Nano 9, 100063 (2020).
41. Kombe, I. K., et al. Model-based estimates of transmission of respiratory syncytial virus within households. Epidemics 27, (2019).
42. Liu, S. L. et al. Single-Virus Tracking: From Imaging Methodologies to Virological Applications. Chemical Reviews vol. 120 Preprint doi: 10.1021/acs.chemrev.9b00692 (2020).
43. Maity, C. K. et al. Induced conducting energy-levels in a boron nitride nano-framework for asymmetric supercapacitors in high charge-mobility ionic electrolytes. Compos B Eng 212, 108728 (2021).
44. Megha, R. et al. Conducting polymer nanocomposite based temperature sensors: A review. Inorg Chem Commun 98, 11-28 (2018).
45. Mikhailov, E., et al. Interaction of aerosol particles composed of protein and saltswith water vapor: hygroscopic growth and microstructural rearrangement. Atmos Chem Phys 4, (2004).
46. Milton, D. K., et al. Influenza Virus Aerosols in Human Exhaled Breath: Particle Size, Culturability, and Effect of Surgical Masks. PLOS Pathog 9, (2013).
47. Mittal, R., et al. The flow physics of COVID-19. J Fluid Mech 894, 1-14 (2020).
48. Nardes, A. M. et al. Microscopic Understanding of the Anisotropic Conductivity of PEDOT:PSS Thin Films. Advanced Materials 19, 1196-1200 (2007).
49. Nardes, A. M., et al. Anisotropic hopping conduction in spin-coated PEDOT:PSS thin films. Phys Rev B 76, 085208 (2007).
50. Nguyen, P., et al. Intergrain Diffusion of Carbon Radical for Wafer-Scale, Direct Growth of Graphene on Silicon-Based Dielectrics. ACS Appl Mater Interfaces 10, acsami.8b07655 (2018).
51. Perasso, A. An Introduction to The Basic Reproduction Number in Mathematical Epidemiology. ESAIM Proc Surv 62, (2018).
52. Perchetti, G. A., et al. Analytical sensitivity of the abbott BinaxNOW COVID-19 ag card. J Clin Microbiol 59, (2021).
53. Putlyaeva, L. V. et al. Studying sars-cov-2 with fluorescence microscopy. International Journal of Molecular Sciences vol. 22 Preprint doi: 10.3390/ijms22126558 (2021).
54. Rai, P. Detection technologies and recent developments in the diagnosis.pdf. 441-455 (2021).
55. Sappia, L. D. et al. PEDOT-polyamine composite films for bioelectrochemical platforms-flexible and easy to derivatize. Materials Science and Engineering C 109, (2020).
56. Seminara, G. et al. Biological fluid dynamics of airborne COVID-19 infection. Rend Lincei Sci Fis Nat 31, 505-537 (2020).
57. Siddique, A. et al. Unprecedented environmental and energy impacts and challenges of COVID-19 pandemic. Environ Res 193, 110443 (2021).
58. Sreeprasad, T. S. et al. Electron-Tunneling Modulation in Percolating Network of Graphene Quantum Dots: Fabrication, Phenomenological Understanding, and Humidity/Pressure Sensing Applications. Nano Lett 13, 1757-1763 (2013).
59. Sui, G. et al. Microfluidics for detection of airborne pathogens: What challenges remain? Bioanalysis 6, 5-7 (2014).
60. Udugama, B. et al. Diagnosing COVID-19: The Disease and Tools for Detection. ACS Nano 14, 3822-3835 (2020).
61. van Doremalen, N. et al. Aerosol and Surface Stability of SARS-CoV-2 as Compared with SARS-CoV-1. New England Journal of Medicine 382, (2020).
62. Verschuur, J., et al. Global economic impacts of COVID-19 lockdown measures stand out in highfrequency shipping data. PLOS One 16, (2021).
63. Wagner, M. M. et al. The emerging science of very early detection of disease outbreaks. Journal of Public Health Management and Practice 7, (2001).
64. Walz, A. L., et al. Commentary regarding: “Activity determination of FAD-dependent glucose dehydrogenase immobilized in PEDOT:PSS-PVA composite films for biosensor applications”. Engineering in Life Sciences vol. 19 Preprint doi: 10.1002/elsc.201900114 (2019).
65. Wang, C., et al. Temperature dependent device characteristics of graphene/h-BN/Si heterojunction. Semicond Sci Technol 35, (2020).
66. Wang, Q. et al. Structural and Functional Basis of SARS-CoV-2 Entry by Using Human ACE2. Cell 181, (2020).
67. Wang, Y. et al. Modeling the effects of timing parameters on virus propagation, in WORM'03 Proceedings of the 2003 ACM Workshop on Rapid Malcode (2003). doi: 10.1145/948187.948198.
68. Williams, T. C., et al. Transmission of paediatric respiratory syncytial virus and influenza in the wake of the COVID-19 pandemic. Euro Surveill 26, (2021).
69. Wu, N. C. et al. Influenza hemagglutinin structures and antibody recognition. Cold Spring Harb Perspect Med 10, (2020).
70. Xu, G. et al. CFD modeling of particle dispersion and deposition coupled with particle dynamical models in a ventilated room. Atmos Environ 166, 300-314 (2017).
71. Yan, J. et al. Infectious virus in exhaled breath of symptomatic seasonal influenza cases from a college community. Proc Natl Acad Sci USA 115, (2018).
72. Zimbovskaya, N. A., et al. Electronic transport mechanism in conducting polymer nanofibers. Phys Rev B 72, 024213 (2005).

What is claimed is:

1. An aerial viral detection device comprising:
a sensor cartridge comprising at least one sensor, the at least one sensor comprising a surface comprising a hydrating component and a conductive component comprising an integrated conductive polymer, integrated nanoparticles, or both an integrated conductive polymer and integrated nanoparticles.

2. The aerial viral detection device of claim 1, wherein the hydrating component comprises the integrated conductive polymer, an external polyelectrolyte, or any combination thereof.

3. The aerial viral detection device of claim 2, wherein the external polyelectrolyte comprises polyallyl hydrochloride.

4. The aerial viral detection device of claim 1, wherein the integrated conductive polymer comprises poly(3,4-ethylenedioxythiophene) polystyrene sulfonate (PEDOT:PSS), polyacetylene (PA), polypyrrole (PPy), poly(p-phenylene) (PPP), or any combination thereof.

5. The aerial viral detection device of claim 1, further comprising a binding agent, the binding agent binding the conductive component to the surface.

6. The aerial viral detection device of claim 1, wherein the nanoparticles comprise silver nanoparticles, gold nanoparticles, carbon-based nanoparticles, or any combination thereof.

7. The aerial viral detection device of claim 1, further comprising a barrier configured to impede conductive electron flow through the aerial viral detection device.

8. The aerial viral detection device of claim 5, further comprising an active element disposed in the sensor cartridge, wherein the active element comprises a molecular receptor disposed in one or more of the binding agent, conductive component, or hydrating component.

9. The aerial viral detection device of claim 8, wherein the molecular receptor comprises one or more antibodies, wherein each of the one or more antibodies binds to at least one viral analyte.

10. The aerial viral detection device of claim 9, wherein the one or more antibodies comprise a SARS CoV-2 antibody, an influenza A53 antibody, a respiratory syncytial virus antibody, or any combination thereof.

11. The aerial viral detection device of claim 9, further comprising a control element disposed in the sensor cartridge, wherein the control element lacks the molecular receptor but is otherwise identical to the active element.

12. The aerial viral detection device of claim 8, further comprising a device body housing the sensor cartridge and one or more of a dust filter connected to the device body, a fan connected to the device body, and a liquid crystal display mounted on an exterior of the device body.

13. The aerial viral detection device of claim 1, further comprising a microcontroller configured to continuously monitor average resistance across the at least one sensor.

14. The aerial viral detection device of claim 1, further comprising electrode contacts in the sensor cartridge, wherein the electrode contacts are connected to the surface of the sensor.

15. The aerial viral detection device of claim 12, further comprising an aerodynamic conical conduit disposed in the device body and configured to direct air flow onto the sensor cartridge.

16. The aerial viral detection device of claim 7, wherein the barrier comprises one or more of an electron or hole transport barrier, carrier activation barrier, or carrier transition barrier.

17. The aerial viral detection device of claim 11, wherein a responsive output of the aerial viral detection device comprises a comparison of a change in resistance at each point of the active element relative the control element.

18. The aerial viral detection device of claim 1, further comprising a cooling element operatively connected to the sensor cartridge, wherein the cooling element enhances viral analyte adsorption.

19. The aerial viral detection device of claim 12, further comprising a vent disposed in the device body and located in a position to increase interaction time of air flowing through the device within the device body.

20. The aerial viral detection device of claim 19, wherein the active element opposes the position of the vent so as to maximize a distance between the vent and the active element.
